(19) United States
(12) Patent Application Publication
(10) Pub. No.: US 2007/0275135 A1
(43) Pub. Date: Nov. 29, 2007

(54) Taste sampling process and product

(75) Inventors: Adnan A. Aziz, Philadelphia, PA (US); Joshua M. Kopelman, Gladwyne, PA (US); Jay B. Minkoff, Wynnewood, PA (US)

Correspondence Address: Daniel H. Golub, 1701 Market Street, Philadelphia, PA, 19103, US

(73) Assignee: First Flavor, Inc., West Conshohocken (PA)

(21) Appl. No.: 11/054,668

(22) Filed: Feb. 9, 2005

(51) Int. Cl. G01N 33/02

(52) U.S. Cl. 426/231

(57) ABSTRACT

A method for marketing a target product having a flavor to a consumer. A consumable film that is distinct from the target product is provided to the consumer using at least one of the following channels: a print advertisement of the target product, a direct mail advertisement of the target product, a print media insert advertising the target product, a dispenser located at a point-of-purchase of the consumer, a display located at the point-of-purchase of the consumer, a coupon for the target product, a channel for test marketing the target product, an advertisement of the target product contained within a package that includes a product different from the product, or an advertisement of the target product attached to the exterior of a package that contains a different product. The consumable film contains at least one water soluble polymer. The consumable film adheres and dissolves in the mouth of the consumer and thereby renders a likeness of the flavor of the target product to the consumer.

### FIELD OF THE INVENTION

The present application is directed to methods and products for sample marketing the flavor of a food or beverage product.

### BACKGROUND OF THE INVENTION

The food and beverage industry is a giant one, with receipts exceeding $587 billion in 2004 and ad spending topping $6 billion. Competition in the food industry is fierce and marketing budgets are ever increasing. However there is a growing trend of unhappiness amongst manufacturers over what is perceived to be a wasting of ad dollars. Most ad dollars are spent on television air time and other visual media. This strategy can only have limited effectiveness due to the fact that ability to process visual information is significantly reduced after 50-55% of total information is visually processed. This points to the fact that sensory marketing, or marketing forms that rely on non-visual stimuli are very important and useful in marketing products.

Another very effective marketing strategy is that of sampling. Actually providing consumers a sample of a product which they have not previously purchased, significantly increases the likelihood of their purchasing that product. It is reported that 85% of consumers buy a new product because of a sample, compared to 67% because of a television advertisement. In addition, 71% of shoppers gave sampling as the main reason for brand switching. A product that could bring the dimension of taste sampling to food and beverage industry marketing is expected to be very well received.

Until now, no marketing method has existed for food and beverage manufacturers, distributors and retailers to use cost effective sample marketing for their products. While traditional sampling done in person does exist, it is prohibitively expensive in many cases and usually not effective enough unless done in a point-of-purchase environment. What is missing in the market is an inexpensive sampling method that food and beverage marketers can use to get their products into the mouths and minds of potential customers.

### SUMMARY OF THE INVENTION

The present invention is directed to a method for marketing a target product having a flavor to a consumer. A consumable film that is distinct from the target product is provided to the consumer using at least one of the following channels: a print advertisement of the target product, a direct mail advertisement of the target product, a print media insert advertising the target product, a dispenser located at a point-of-purchase of the consumer, a display located at the point-of-purchase of the consumer, a coupon for the target product offered to the consumer, a channel for test marketing the target product, an advertisement of the target product contained within a package that includes a different product, or an advertisement of the target product attached to the exterior of a package that contains a different product. The consumable film contains at least one water soluble polymer. The consumable film adheres and dissolves in the mouth of the consumer and thereby renders a likeness of the flavor of the target product to the consumer.

The consumable film optionally includes an endothermic agent that delivers a cooling sensation to the consumer when the consumable film adheres and dissolves in the mouth of the consumer; an exothermic agent that delivers a heating sensation to the consumer when the consumable film adheres and dissolves in the mouth of the consumer; and/or at least one encapsulated agent that delivers a fizzing sensation to the consumer when the consumable film adheres and dissolves in the mouth of the consumer. In one embodiment, the fizzing sensation is created by a reaction of a carbon dioxide source and an acid source.

In accordance with a further aspect, the present invention is directed to a method for marketing a target product having a flavor to consumers. Flavor information about the target product is received from a party desiring to market the product. The flavor information is used to manufacture a plurality of individual consumable film strips that are distinct from the target product. The individual consumable film strips are provided to the consumers. Each of the individual consumable film strips contains at least one water soluble polymer, and each of the consumable film strips adheres and dissolves in a mouth of the consumer and thereby renders a likeness of the flavor of the target product to the consumer.

In accordance with a still further aspect, the present invention is directed to a printed medium for marketing a target product having a flavor to a consumer. At least one consumable film strip is fastened to the printed medium. The consumable film strip is distinct from the target product, and contains at least one water soluble polymer. The consumable film strip adheres and dissolves in the mouth of the consumer to thereby render a likeness of the flavor of the target product to the consumer. The printed medium further contains a printed advertising message associated with the target product. Examples of the printed medium include a magazine advertisement and a direct mail advertisement of the target product.

### BRIEF DESCRIPTION OF THE DRAWINGS

FIG. 1 is a flow diagram illustrating a method for marketing a target product having a flavor to a consumer, in accordance with the present invention.

FIG. 2 is a diagram illustrating a consumable film strip individually packaged in a wrapper, in accordance with the present invention.

FIG. 3 is a diagram illustrating a printed medium such as a magazine advertisement or direct mail advertisement for marketing a target product having a flavor to a consumer, in accordance with the present invention.

FIG. 4 illustrates a pouch for packaging and distributing an edible film strip in accordance with the present invention, wherein the pouch contains an opening for attaching the pouch to the neck of a bottle.

FIG. 5 illustrates a pouch 500 for packaging and distributing an edible film strip in accordance with the present invention, wherein the pouch contains an opening for hanging the pouch (with the film strip contained therein) at the point-of-purchase of the target product.

### DETAILED DESCRIPTION OF THE PREFERRED EMBODIMENT

The present invention is a new method of marketing, which brings taste to the marketing arena. The inventive process includes replication of a target flavor on the readily transferable and consumer friendly medium of edible film strips. The process also provides opportunities to add in special effects which help to further mimic the product experience, such as an endothermic reaction to give a cooling effect, or an exothermic effect to produce a heating effect, or a acid-base reaction to produce a fizzing effect. The present invention permits food and beverage manufacturers and marketers to inexpensively sample market the flavor of their products to consumers.

Consider, by way of example, a situation where a beverage company is interested in using the present invention to market its cola soda. Using the marketing method of the present invention, the flavor of the cola soda is replicated in an edible film strip, including introduction of endothermic and fizz effects into the edible film strip. When a consumer puts this strip into their mouth, they taste a cold, fizzing replication of the cola flavored soda. The edible film strips that replicate the cola flavored soda can be distributed and used in a number of different ways for marketing the cola product itself, including: as part of an insert in print advertising, as part of a direct mail piece, in a dispenser at point-of-purchase and as a test marketing piece.

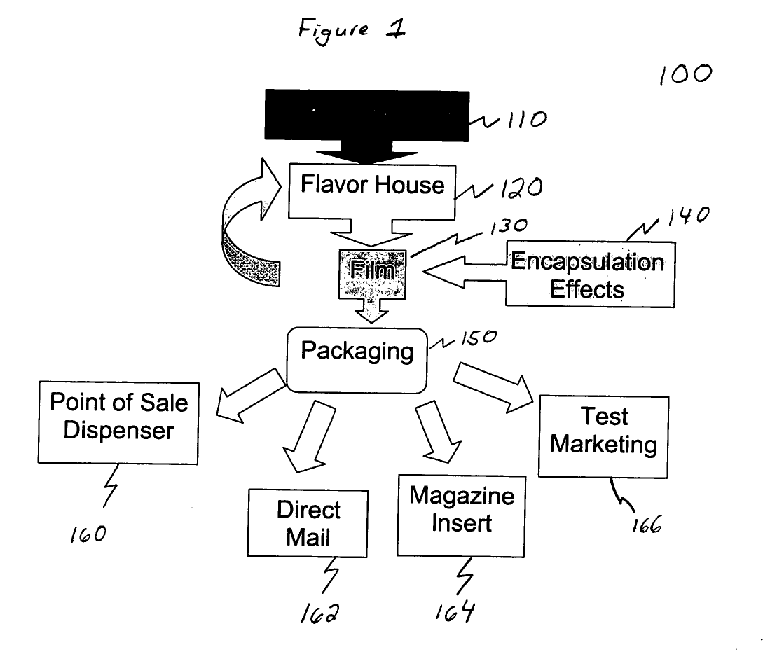

Referring now to FIG. 1, there is shown a flow diagram illustrating a method 100 for marketing a target product (e.g., a cola flavored soda, or any other food or beverage) having a flavor to a consumer, in accordance with the present invention. In step 110, flavor information about the target product is received from a party (e.g., a manufacturer or distributor of the target product) desiring to market the target product. In one embodiment, the flavor information includes a recipe of the actual flavor mix used to manufacture the target product. In step 120, the flavor information (e.g., the flavor mix) is used to generate a compound that can be placed into an edible film such that, when the edible film dissolves in the mouth of the consumer, the edible film will render a likeness of the flavor of the target product to the consumer. Methods for generating such compounds for edible film strips that will render a likeness of the flavor of the target product are well known in the art. Some such methods are taught, for example, in U.S. Pat. No. 6,834,530 to Kita, et al. and US Patent Application 2004/0260617 to Davidson, et al., which are incorporated herein in their entirety by reference, and at the following websites: http://www.fks.com/flavors/tech/Science%20of%20Flavor%20Creation.asp; http://www.preparedfoods.com/CDA/ArticleInformation/coverstory/BNPCoverStoryltem/0,1229,113836,00.html.

In step 130, a plurality of individual edible film strips are manufactured by including a suitable amount of the compound generated in step 120 on each of the edible film strips. Methods for generating suitable edible film strips are well known in the art and are taught, for example, in U.S. Pat. Nos. 6,824,829 to Berry, et al., 6,596,298 to Leung, et al., and 6,824,829 to Berry, et al., all of which are incorporated herein by reference, and in US Patent Applications 20030054034 to Leung, et al., 20030211136 to Kulkarni, et al., 20040180110 to Mistry, 20040202698 to Ramji, et al., 20040223921 to Rau, et al., 20040247746 to Pearce, et al., 20040081680 to Pesce, et al., 20040043134 to Corriveau, et al., and 20030224090 to Pearce, et al., all which are incorporated herein in their entirety by reference. Each of the individual consumable film strips generated in step 130 are distinct from the target product, contain at least one water soluble polymer, and each of the consumable film strips adheres and dissolves in the mouth of the consumer and thereby renders a likeness of the flavor of the target product to the consumer. Examples of suitable water soluble polymers for use with the present invention include pullulan, hydroxyproplymethyl cellulose, hydroxyethyl cellulose, ethylhydroxyethyl cellulose, methyl cellulose, hydroxypropyl cellulose, polyvinyl pyrrolidone, carboxymethyl cellulose, salts of carboxymethyl cellulose, polyvinyl alcohol, sodium alginate, polyethylene glycol, tragacanth gum, guar gum, acacia gum, natural seed gum, locust gum, tara gum, ghatti gum, agar gum and zanthan gum, Arabic gum, polyacrylic acid, methylmethacrylate copolymer, carboxyvinyl polymer, amylase, high amylase starch, hydroxypropylate high amylase starch, dextrin, pectin, chitin, chitosan, levan, elsinan, collagen, gelatin, zein, gluten, soy protein isolate, whey protein isolate, casein, calcium alginate, carrageenan, a maltodextrin and mixtures thereof.

Optionally, in step 140, other information about effects caused by the target product in the mouth of the consumer is received from the party desiring to market the target product. In one embodiment, the effect information includes a temperature effect (e.g., cold or hot) or a fizzing effect (e.g., effervescence) caused by the target product in the mouth of the consumer. In step 140, this effect information is used to generate one or more other compounds that can be placed into the edible film strip such that, when the edible film dissolves in the mouth of the consumer, the edible film will render a likeness of an effect (e.g., cold, hot, fizzing) of the target product to the consumer. Examples of endothermic agents that can be included in the edible film strips and used to deliver a cooling sensation to the consumer when the consumable film adheres and dissolves in the mouth of the consumer include sugar alcohol and polyhydric alcohol. Aromatic aledhyde is an example of an exothermic agent that can be included in the edible film strips and used to deliver a heating sensation to the consumer when the consumable film adheres and dissolves in the mouth of the consumer. Flavoring agents may also be included in the consumable film to produce a cooling or warming sensation to the consumer when the consumable film adheres and dissolves in the mouth of the consumer. Examples of such flavoring agents that may be used to provide a cooling sensation include menthol, menthyl lactate, menthyl ethoxyacetate, menthone glycerinacetal, 3-1-menthoxypropane-1,2-diol, ethyl 1-menthyl carbonate, (1S,3S,4R)-p-menth-8-en-3-ol, menthyl pyrrolidone carboxylate, N-substituted-p-menthane-3-carboxamides N-ethyl-p-menthane-3-carboxamide, acyclic carboxamides, N,2,3-trimethyl-2-isopropylbutanamide, ketal coolants, 0.02-0.06 ppm 4-methyl-3-(1-pyrrolidinyl)-2[5H]-furanone, 1.5-3.0 ppm 5-methyl-4-(1-pyrrolidinyl)-3-[2H]-furanone, 2.0-4.0 ppm 4,5-dimethyl-3-(1-pyrrolidinyl)-2[5H]-furanone, and 1-menthon-1d-isomenthon glycerin ketal; flavoring agents that may be used to provide a warming sensation include (E)-ocimene, capsaicin, piperine, and isothiocyanates. By way of further example, encapsulated agents can be included in the edible film strips and used to deliver a fizzinge sensation to the consumer when the consumable film adheres and dissolves in the mouth of the consumer. The fizzing sensation may be created by a reaction of a carbon dioxide source (e.g., sodium bicarbonate, sodium carbonate, potassium bicarbonate, potassium carbonate, calcium carbonate, magnesium carbonate and mixtures thereof) and an acid source (e.g., citric acid, fumaric acid, tartaric acid, malic acid, adipic acid and mixtures thereof.)

In step 150, each of the individual consumable film strips are packaged and distributed to consumers using one or more of a variety of different marketing channels including a dispenser located at a point-of-purchase of the consumer (step 160), a direct mail advertisement of the target product (step 162), a print advertisement of the target product (step 164), or a channel for test marketing the target product (step 166). Film strips for use in point of purchase films (step 160) are preferably packaged in spools and sent out to fit into a point of purchase dispenser. Film strips for use in a direct mail advertisement of the target product (step 162), a print advertisement of the target product (step 164), or a channel for test marketing the target product (step 166) are preferably individually packaged.

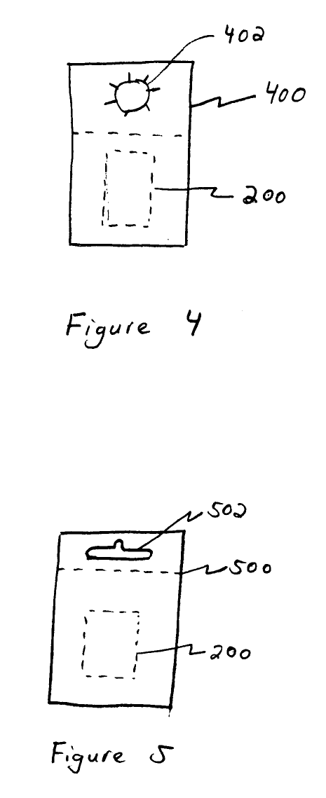

Alternatively, the individual consumable film strips may be packaged and distributed to consumers as an advertisement of the target product that is contained within a package that includes a different product. For example, one or more individually packaged film strips may be included as a give-away in a box of cereal, in order to promote a target food or beverage product that is different from the cereal. In a still further embodiment, the individual consumable film strips may be packaged and distributed to consumers as an advertisement of the target product that is contained on the outside of a package that contains a different product. For example, one or more individually packaged film strips may be included as a give-away that is attached to the neck of a soda bottle, in order to promote a target food or beverage product that is different from the soda contained in the bottle. FIG. 4 illustrates a pouch 400 for packaging and distributing a film strip in accordance with this embodiment of the present invention. Pouch 400 contains an opening 402 for attaching the pouch to the neck of a bottle (not shown).

A print media insert advertising the target product represents another marketing channel through which the individual consumable film strips may be packaged and distributed to consumers. For example, individual consumable film strips may mounted on card stock and then wrapped in order to create inserts to magazines and newspapers. The card stock, or other literature associated with the insert will contain printed information promoting the target product.

A display located at the point-of-purchase of the consumer represents another marketing channel through which the individual consumable film strips may be packaged and distributed to consumers. For example, individual consumable film strips may wrapped or contained in pouches that are hung at the point-of-purchase in order to create a promotion for the target product. FIG. 5 illustrates a pouch 500 for packaging and distributing a film strip in accordance with this embodiment of the present invention. Pouch 500 contains an opening 502 for hanging the pouch from a hook or rod (not shown) located at the point-of-sale.

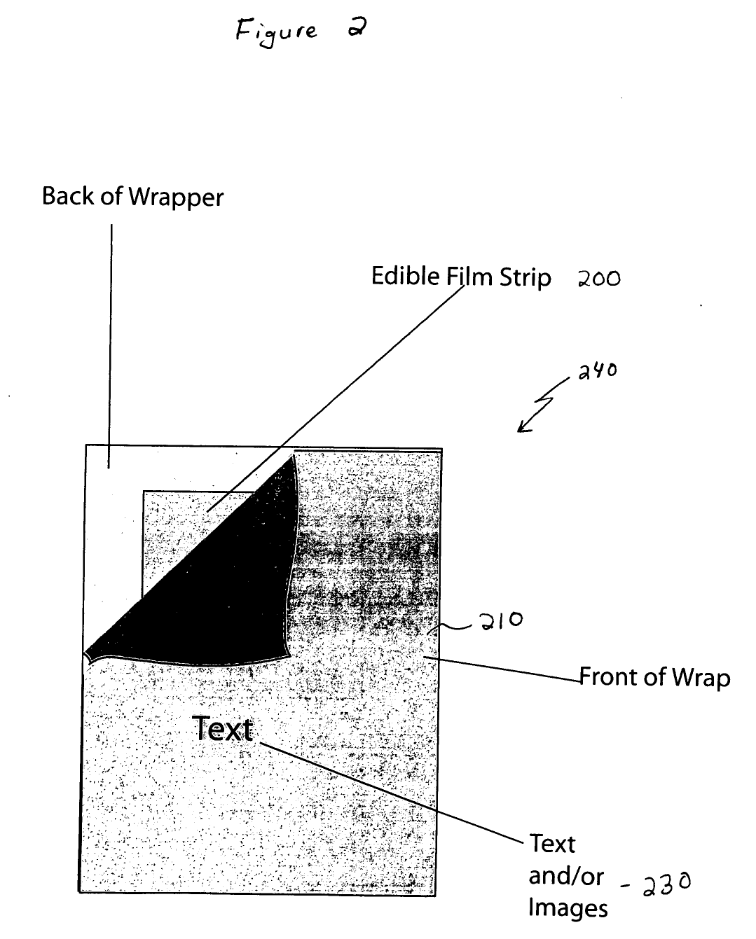

Referring now to FIG. 2, there is a diagram illustrating a consumable film strip 200 formed as described above and individually packaged in a wrapper 210, in accordance with the present invention. The wrapper 210 may be formed from any suitable material including polyethylene, polystyrene, polypropylene, polyamide, polyester, polyolefin, mylar, aluminum foil, and cellulose, and each edible film strip may be packaged therein using known automated packaging technology. Wrapper 210 may include text or images 230 associated with the target product. In one embodiment, each edible film strip is about 0.75-1.0″×1.0-1.5″, and more preferably 0.875″×1.25″, in size.

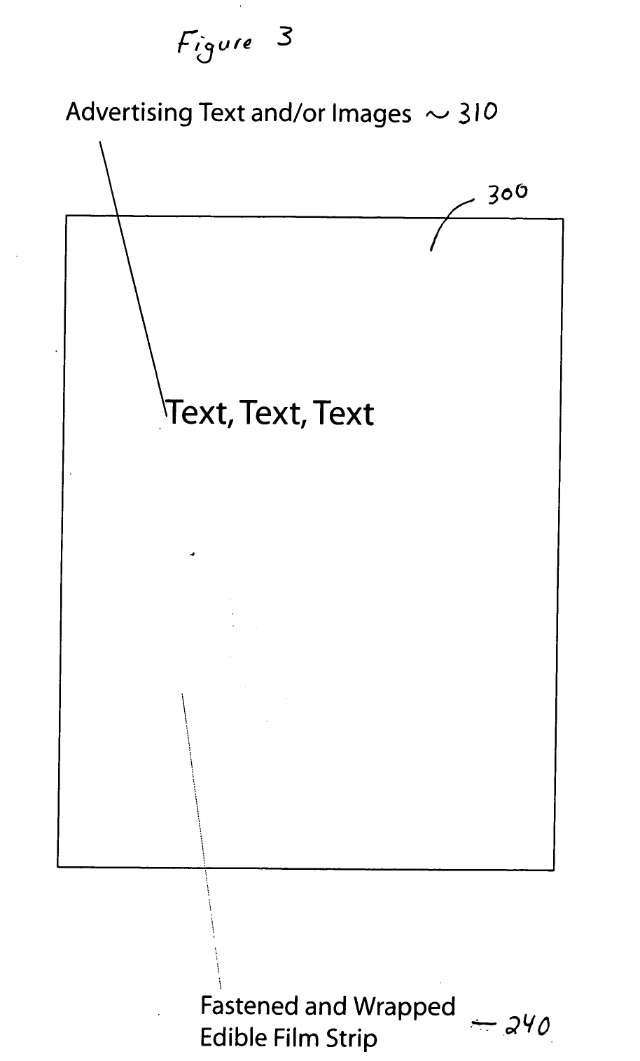

Referring now to FIG. 3, there is a diagram illustrating a printed medium 300 such as a magazine/newspaper advertisement or insert, point-of-purchase display or direct mail advertisement for marketing a target product having a flavor to a consumer, in accordance an embodiment of the present invention. At least one wrapped consumable film strip 240 is linked or fastened to the printed medium 300. The printed medium 300 further contains a printed advertising message 310 associated with the target product. Alternatively, the printed medium also contains or is linked to a store coupon offered to the consumer, that may be used by the consumer to purchase the target product at a discount.

Finally, it will be appreciated by those skilled in the art that changes could be made to the embodiments described above without departing from the broad inventive concept thereof. It is understood, therefore, that this invention is not limited to the particular embodiments disclosed, but is intended to cover modifications within the spirit and scope of the present invention as defined in the appended claims.

What is claimed is:

1. A method for marketing a target product having a flavor to a consumer, comprising:
providing a consumable film to the consumer, wherein the consumable film is distinct from the target product, the consumable film contains at least one water soluble polymer, and the consumable film adheres and dissolves in a mouth of the consumer and thereby renders a likeness of the flavor of the target product to the consumer;
wherein the consumable film is provided to the consumer using at least one of the following channels: a print advertisement of the target product, a direct mail advertisement of the target product, a print media insert advertising the target product, a dispenser located at a point-of-purchase of the consumer, a display located at the point-of-purchase of the consumer, a coupon for the target product offered to the consumer, a channel for test marketing the target product, an advertisement of the target product contained within a package that includes a different product, or an advertisement of the target product affixed to an exterior of a package that contains a different product.

2. The method of claim 1, wherein the water soluble polymer is selected from the group consisting of pullulan, hydroxyproplymethyl cellulose, hydroxyethyl cellulose, ethylhydroxyethyl cellulose, methyl cellulose, hydroxypropyl cellulose, polyvinyl pyrrolidone, carboxymethyl cellulose, salts of carboxymethyl cellulose, polyvinyl alcohol, sodium alginate, polyethylene glycol, tragacanth gum, guar gum, acacia gum, natural seed gum, locust gum, tara gum, ghatti gum, agar gum and zanthan gum, Arabic gum, polyacrylic acid, methylmethacrylate copolymer, carboxyvinyl polymer, amylase, high amylase starch, hydroxypropylate high amylase starch, dextrin, pectin, chitin, chitosan, levan, elsinan, collagen, gelatin, zein, gluten, soy protein isolate, whey protein isolate, casein, calcium alginate, carrageenan, a maltodextrin and mixtures thereof.

3. The method of claim 1, wherein the consumable film further comprises one or more flavoring agents that reproduce the flavor of the target product to the consumer when the consumable film dissolves in the mouth of the consumer.

4. The method of claim 3, wherein at least one of the flavoring agents is encapsulated.

5. The method of claim 1, wherein the consumable film contains an endothermic agent that delivers a cooling sensation to the consumer when the consumable film adheres and dissolves in the mouth of the consumer.

6. The method of claim 5, wherein the endothermic agent is selected from the group consisting of a sugar alcohol and a polyhydric alcohol.

7. The method of claim 1, wherein the consumable film contains an exothermic agent that delivers a heating sensation to the consumer when the consumable film adheres and dissolves in the mouth of the consumer.

8. The method of claim 7, wherein the exothermic agent is an aromatic aledhyde.

9. The method of claim 1, wherein the consumable film contains a flavoring agent that delivers a cooling sensation to the consumer when the consumable film adheres and dissolves in the mouth of the consumer.

10. The method of claim 1, wherein the consumable film contains a flavoring agent that delivers a heating sensation to the consumer when the consumable film adheres and dissolves in the mouth of the consumer.

11. The method of claim 1, wherein the consumable film contains at least one encapsulated agent that delivers a fizzing sensation to the consumer when the consumable film adheres and dissolves in the mouth of the consumer.

12. The method of claim 11, wherein the fizzing sensation is created by a reaction of a carbon dioxide source and an acid source.

13. The method of claim 12, wherein the carbon dioxide source is selected from the group consisting of sodium bicarbonate, sodium carbonate, potassium bicarbonate, potassium carbonate, calcium carbonate, magnesium carbonate and mixtures thereof.

14. The method of claim 12, wherein the acid source is selected from the group consisting of citric acid, fumaric acid, tartaric acid, malic acid, adipic acid and mixtures thereof.

15. A method for marketing a target product having a flavor to consumers, comprising:
receiving flavor information about the target product from a party desiring to market the product;
using the flavor information to manufacture a plurality of individual consumable film strips that are distinct from the target product; and
providing the individual consumable film strips to the consumers, wherein each of the individual consumable film strips contains at least one water soluble polymer, and each of the consumable film strips adheres and dissolves in a mouth of a consumer and thereby renders a likeness of the flavor of the target product to the consumer.

16. The method of claim 15, wherein the water soluble polymer is selected from the group consisting of pullulan, hydroxyproplymethyl cellulose, ethylhydroxyethyl cellulose, methyl cellulose, hydroxyethyl cellulose, hydroxypropyl cellulose, polyvinyl pyrrolidone, carboxymethyl cellulose, salts of carboxymethyl cellulose, polyvinyl alcohol, sodium alginate, polyethylene glycol, tragacanth gum, guar gum, acacia gum, natural seed gum, locust gum, tara gum, ghatti gum, agar gum and zanthan gum, Arabic gum, polyacrylic acid, methylmethacrylate copolymer, carboxyvinyl polymer, amylase, high amylase starch, hydroxypropylate high amylase starch, dextrin, pectin, chitin, chitosan, levan, elsinan, collagen, gelatin, zein, gluten, soy protein isolate, whey protein isolate, casein, calcium alginate, carrageenan, a maltodextrin and mixtures thereof.

17. The method of claim 15, wherein each consumable film strip further comprises one or more flavoring agents that reproduce the flavor of the target product to the consumer when the consumable film dissolves in the mouth of the consumer.

18. The method of claim 17, wherein at least one of the flavoring agents is encapsulated.

19. The method of claim 15, wherein each of the consumable film strips contains an endothermic agent that delivers a cooling sensation to the consumer when the consumable film strip adheres and dissolves in the mouth of the consumer.

20. The method of claim 19, wherein the endothermic agent is selected from the group consisting of a sugar alcohol and a polyhydric alcohol.

21. The method of claim 15, wherein each of the consumable film strips contains an exothermic agent that delivers a heating sensation to the consumer when the consumable film strip adheres and dissolves in the mouth of the consumer.

22. The method of claim 21, wherein the exothermic agent is an aromatic aledhyde.

23. The method of claim 15, wherein the consumable film contains a flavoring agent that delivers a cooling sensation to the consumer when the consumable film adheres and dissolves in the mouth of the consumer.

24. The method of claim 15, wherein the consumable film contains a flavoring agent that delivers a heating sensation to the consumer when the consumable film adheres and dissolves in the mouth of the consumer.

25. The method of claim 15, wherein each of the consumable film strips contains at least one encapsulated agent that delivers a fizzing sensation to the consumer when the consumable film strip adheres and dissolves in the mouth of the consumer.

26. The method of claim 25, wherein the fizzing sensation is created by a reaction of a carbon dioxide source and an acid source.

27. The method of claim 26, wherein the carbon dioxide source is selected from the group consisting of sodium bicarbonate, sodium carbonate, potassium bicarbonate, potassium carbonate, calcium carbonate, magnesium carbonate and mixtures thereof.

28. The method of claim 26, wherein the acid source is selected from the group consisting of citric acid, fumaric acid, tartaric acid, malic acid, adipic acid and mixtures thereof.

29. A printed medium for marketing a target product having a flavor to a consumer, comprising:
at least one consumable film strip linked to the printed medium, wherein the consumable film strip is distinct from the target product, the consumable film strip contains at least one water soluble polymer, and the consumable film strip adheres and dissolves in a mouth of the consumer to thereby render a likeness of the flavor of the target product to the consumer;
wherein the printed medium further contains a printed advertising message associated with the target product.

30. The printed medium of claim 29, wherein the printed medium corresponds to at least one of: a magazine advertisement, a newspaper advertisement, a magazine insert, or a newspaper insert.

31. The printed medium of claim 29, wherein the printed medium corresponds to a direct mail advertisement.

32. The printed medium of claim 29, wherein the printed medium corresponds to an advertisement of the target product that is contained within a package that includes a product that is different from the target product.

33. The printed medium of claim 29, wherein the printed medium corresponds to an advertisement of the target product that is attached to the outside of a package that includes a product that is different from the target product.

34. The printed medium of claim 33, wherein the printed medium includes an opening for attaching the printed medium to a neck of a bottle.

35. The printed medium of claim 29, wherein the printed medium corresponds to a point of purchase display.

36. The printed medium of claim 35, wherein the printed medium includes an opening for hanging the printed medium from a rod or hook at a point of purchase of the target product.

37. The printed medium of claim 29, wherein the printed medium includes a coupon for purchasing the target product at a discount price.

38. The printed medium of claim 29, wherein the water soluble polymer is selected from the group consisting of pullulan, hydroxyproplymethyl cellulose, hydroxyethyl cellulose, ethylhydroxyethyl cellulose, methyl cellulose, hydroxypropyl cellulose, salts of carboxymethyl cellulose, polyvinyl pyrrolidone, carboxymethyl cellulose, polyvinyl alcohol, sodium alginate, polyethylene glycol, tragacanth gum, guar gum, acacia gum, natural seed gum, locust gum, tara gum, ghatti gum, agar gum and zanthan gum, Arabic gum, polyacrylic acid, methylmethacrylate copolymer, carboxyvinyl polymer, amylase, high amylase starch, hydroxypropylate high amylase starch, dextrin, pectin, chitin, chitosan, levan, elsinan, collagen, gelatin, zein, gluten, soy protein isolate, whey protein isolate, casein, calcium alginate, carrageenan, a maltodextrin and mixtures thereof.

39. The printed medium of claim 29, wherein the consumable film strip further comprises one or more flavoring agents that reproduce the flavor of the target product to the consumer when the consumable film strip dissolves in the mouth of the consumer.

40. The printed medium of claim 39, wherein at least one of the flavoring agents is encapsulated.

41. The printed medium of claim 29, wherein the consumable film strip contains an endothermic agent that delivers a cooling sensation to the consumer when the consumable film strip adheres and dissolves in the mouth of the consumer.

42. The printed medium of claim 41, wherein the endothermic agent is selected from the group consisting of a sugar alcohol and a polyhydric alcohol.

43. The printed medium of claim 29, wherein the consumable film strip contains an exothermic agent that delivers a heating sensation to the consumer when the consumable film strip adheres and dissolves in the mouth of the consumer.

44. The printed medium of claim 43, wherein the exothermic agent is an aromatic aledhyde.

45. The printed medium of claim 29, wherein the consumable film strip contains at least one encapsulated agent that delivers a fizzing sensation to the consumer when the consumable film strip adheres and dissolves in the mouth of the consumer.

46. The printed medium of claim 45, wherein the fizzing sensation is created by a reaction of a carbon dioxide source and an acid source.

47. The printed medium of claim 46, wherein the carbon dioxide source is selected from the group consisting of sodium bicarbonate, sodium carbonate, potassium bicarbonate, potassium carbonate, calcium carbonate, magnesium carbonate and mixtures thereof.

48. The printed medium of claim 46, wherein the acid source is selected from the group consisting of citric acid, fumaric acid, tartaric acid, malic acid, adipic acid and mixtures thereof.

49. The printed medium of claim 29, wherein the consumable film contains a flavoring agent that delivers a cooling sensation to the consumer when the consumable film adheres and dissolves in the mouth of the consumer.

50. The printed medium of claim 29, wherein the consumable film contains a flavoring agent that delivers a heating sensation to the consumer when the consumable film adheres and dissolves in the mouth of the consumer.
